(19) United States
(12) Patent Application Publication
(10) Pub. No.: US 2026/0276664 A1
(43) Pub. Date: Sep. 17, 2026

(54) AUTOMATIC ANALYSIS APPARATUS

(71) Applicant: CANON MEDICAL SYSTEMS CORPORATION, Otawara-shi (JP)

(72) Inventor: Shozo HASHIMOTO, Nasushiobara (JP)

(73) Assignee: CANON MEDICAL SYSTEMS CORPORATION, Otawara-shi (JP)

(21) Appl. No.: 19/563,810

(22) Filed: Mar. 11, 2026

(30) Foreign Application Priority Data
Mar. 13, 2025 (JP) 2025-040638

(51) Int. Cl. G01N 35/00

(52) U.S. Cl. CPC G01N 35/00732; G01N 35/00663; G01N 2035/00673; G01N 2035/00851; G01N 2035/0091; G01N 2035/0097

(57) ABSTRACT

An automatic analysis apparatus according to an embodiment analyzes a mixture of a sample and a reagent corresponding to a measurement item for the sample by using the sample and the reagent. The automatic analysis apparatus includes a flag setting unit and a threshold setting unit. The flag setting unit sets a flag for the component increase/decrease item. The flag indicates that the item is a component increase/decrease item. The component increase/decrease item is a measurement item in which a measurement target component in the sample increases or decreases over time. The threshold setting unit sets a first threshold for the component increase/decrease item for which the flag is set. The first threshold relates to a time until an increase or decrease of the measurement target component affects a measurement result.

### CROSS-REFERENCE TO RELATED APPLICATIONS

This application is based upon and claims the benefit of priority from Japanese Patent Application No. 2025-040638, filed Mar. 13, 2025, the entire contents of which are incorporated herein by reference.

### FIELD

Embodiments described herein relate generally to an automatic analysis apparatus.

### BACKGROUND

An automatic analysis apparatus is an apparatus that analyzes a component of a test sample that corresponds to each measurement item, for example, by optically measuring a reaction solution that is obtained by mixing a sample such as a test sample, such as blood, collected from a subject or a standard sample for each measurement item, with a reagent corresponding to each measurement item. When an error occurs or an abnormal measurement value is obtained in a measurement using such an automatic analysis apparatus, there is a need to carry out a remeasurement of a target sample. However, there are cases where it takes time before the remeasurement can be started.

Depending on a measurement target component to be measured using the automatic analysis apparatus, the component may increase or decrease over time. Thus, in a case where the measurement target component that increases or decreases over time is to be measured, there is a need to take into account an elapsed time from the time of blood collection. Furthermore, because the amount of increase or decrease of the measurement target component may vary depending on an environment in which the target sample has been placed even if the elapsed time is the same, it is also necessary to take into account the environment in which the sample has been placed in the case where the measurement target component that increases or decreases over time is to be measured.

However, since the elapsed time from the time of blood collection, the environment in which the sample has been placed, and the like are not taken into account in the measurement of the measurement target component that increases or decreases over time, a probability that an increase or decrease of the measurement target component over time will affect a diagnosis increases. Therefore, there is a demand for reducing the probability that the increase or decrease of the measurement target component over time will affect a diagnosis in the measurement using the automatic analysis apparatus.

### BRIEF DESCRIPTION OF THE DRAWINGS

FIG. 1 is a block diagram illustrating an example of a functional configuration of an automatic analysis apparatus according to a first embodiment;

FIG. 2 is a schematic view illustrating a configuration of an analysis mechanism according to the first embodiment;

FIG. 3 illustrates an example of an item setting screen according to the first embodiment;

FIG. 4 illustrates an example of the item setting screen according to the first embodiment;

FIG. 5 illustrates an example of the item setting screen according to the first embodiment;

FIG. 6 illustrates an example of the item setting screen according to the first embodiment;

FIG. 7 is a flowchart illustrating contents of measurement result output processing executed by the automatic analysis apparatus according to the first embodiment;

FIG. 8 illustrates an example of a measurement result screen;

FIG. 9 illustrates an example of a measurement availability selection screen according to the first embodiment;

FIG. 10 illustrates another example of the measurement availability selection screen according to the first embodiment;

FIG. 11 illustrates an example of the measurement result screen including a measurement result for a component increase/decrease item according to the first embodiment;

FIG. 12 illustrates an example of an item setting screen according to a second embodiment;

FIG. 13 is a flowchart illustrating contents of measurement result output processing executed by an automatic analysis apparatus according to the second embodiment;

FIG. 14 illustrates an example of a measurement result screen including a measurement result including information indicating that a component increase/decrease item has been measured according to the second embodiment;

FIG. 15 illustrates an example of an item setting screen according to a first modified example; and

FIG. 16 illustrates an example of a measurement result screen including a measurement result for a component increase/decrease item according to a second modified example.

### DETAILED DESCRIPTION

An automatic analysis apparatus according to an embodiment analyzes a mixture of a sample and a reagent corresponding to a measurement item for the sample by using the sample and the reagent. The automatic analysis apparatus includes a flag setting unit and a threshold setting unit. The flag setting unit sets a flag for the component increase/decrease item. The flag indicates that the item is a component increase/decrease item. The component increase/decrease item is a measurement item in which a measurement target component in the sample increases or decreases over time. The threshold setting unit sets a first threshold for the component increase/decrease item for which the flag is set. The first threshold relates to a time until an increase or decrease of the measurement target component affects a measurement result.

Various Embodiments will be described hereinafter with reference to the accompanying drawings.

In the following description, components having substantially the same functions and configurations are denoted by the same reference numerals, and redundant explanations are provided only when necessary.

### First Embodiment

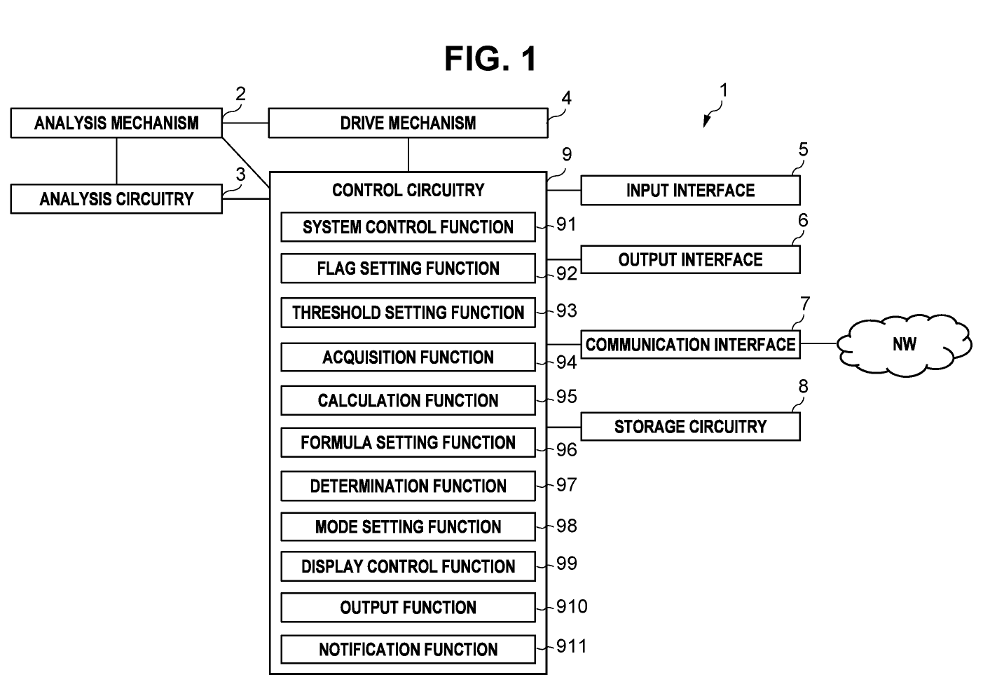

FIG. 1 is a block diagram illustrating an example of a functional configuration of an automatic analysis apparatus according to a first embodiment. An automatic analysis apparatus 1 according to the first embodiment is an apparatus that analyzes a mixture of a sample and a reagent using the sample and the reagent corresponding to the measurement item for the sample. In other words, the automatic analysis apparatus 1 measures components in a sample by measuring a mixture obtained by adding a reagent to the sample to be measured. As illustrated in FIG. 1, the automatic analysis apparatus 1 according to the first embodiment includes, for example, an analysis mechanism 2, analysis circuitry 3, a drive mechanism 4, an input interface 5, an output interface 6, a communication interface 7, storage circuitry 8, and control circuitry 9.

The analysis mechanism 2 adds, to a sample such as a standard sample or a test sample, a reagent corresponding to a measurement item for the sample. The analysis mechanism 2 measures the mixture obtained by adding the reagent to the sample and, for example, generates standard data and test data. In the first embodiment, the standard data represents a result of measuring absorbance of the standard sample in which concentration of a detection target contained therein is known. Furthermore, the test data represents a result of measuring absorbance of the test sample. In the following description, in a case where the standard sample and the test sample are expressed without distinction, these samples may simply be referred to as a "sample".

The analysis circuitry 3 is a processor that analyzes the standard data and the test data generated by the analysis mechanism 2, thereby generating calibration data, analysis data, and the like. The analysis circuitry 3 reads out an analysis program from the storage circuitry 8 and generates the calibration data, analysis data, and the like according to the read analysis program. For example, the analysis circuitry 3 generates standard data, and calibration data indicating a relationship with a preset standard value for a standard sample based on the standard data. Furthermore, the analysis circuitry 3 generates the analysis data that is expressed as a concentration value and an activity value of an enzyme based on the test data and the calibration data on an examination item corresponding to the test data. The analysis circuitry 3 outputs the generated calibration data, analysis data, and the like to the control circuitry 9.

The drive mechanism 4 drives the analysis mechanism 2 under control of the control circuitry 9. The drive mechanism 4 is implemented by, for example, a gear, a stepping motor, a belt conveyor, a lead screw, and the like.

The input interface 5 receives, for example, a setting of an analysis parameter or the like corresponding to each measurement item related to the sample for which measurement has been requested. The input interface 5 is implemented by, for example, a mouse, a keyboard, a touch-pad for which an instruction is input by touching an operation surface, and the like. The input interface 5 is connected to the control circuitry 9, converts an operation instruction input by a user into an electric signal, and outputs the electric signal to the control circuitry 9. In the first embodiment, the input interface 5 is not limited to an interface that includes a physical operation component such as a mouse or a keyboard. For example, electric signal processing circuitry that receives an electric signal corresponding to an operation instruction input from an external input device provided separately from the automatic analysis apparatus 1 and outputs the electric signal to the control circuitry 9 is also included in examples of the input interface 5.

The output interface 6 is connected to the control circuitry 9 and outputs a signal supplied from the control circuitry 9. The output interface 6 is implemented by, for example, display circuitry, print circuitry, an audio device, and the like. Examples of the display circuitry include a cathode ray tube (CRT) display, a liquid crystal display, an organic electroluminescence (EL) display, a light-emitting diode (LED) display, and a plasma display. The display circuitry corresponds to a display unit according to the first embodiment. Processing circuitry that converts data representing a display target into a video signal and outputs the video signal to the outside is also included in the examples of the display circuitry. Examples of the print circuitry include a printer. Output circuitry that outputs data representing a print target to the outside is also included in the examples of the print circuitry. Examples of the audio device include a speaker. Output circuitry that outputs an audio signal to the outside is also included in the examples of the audio device.

The communication interface 7 is connected to, for example, an in-hospital network NW, and connects the automatic analysis apparatus 1 to the in-hospital network NW. The communication interface 7 communicates data with a hospital information system (HIS) via the in-hospital network NW. The communication interface 7 may communicate data with the HIS via a laboratory information system (LIS) connected to the in-hospital network NW.

The storage circuitry 8 includes a processor-readable recording medium such as a magnetic or optical recording medium or a semiconductor memory. The storage circuitry 8 stores an analysis program to be executed by the analysis circuitry 3 and a control program to be executed by the control circuitry 9. The storage circuitry 8 also stores analysis data generated by the analysis circuitry 3 for each measurement item. The storage circuitry 8 also stores examination order information input by an operator, examination order information received by the communication interface 7 via the in-hospital network NW, and the like. The examination order information includes a sample identification (ID) and a measurement item required for the sample to be measured. The storage circuitry 8 further stores measurement item information as described in detail below. The storage circuitry 8 need not necessarily be implemented by a single storage device. The storage circuitry 8 can also be implemented by, for example, a plurality of storage devices.

The measurement item information is information about measurement items. Examples of the measurement item information include a measurement item name, a name of reagent used for each measurement item, the amount of reagent, and various parameters for measurement of the measurement items. The measurement item information further includes information about a component increase/decrease item according to user settings in a case where the measurement item is a component increase/decrease item. The component increase/decrease item is a measurement item in which a measurement target component in the sample to be measured with regard to the measurement item increases or decreases over time. Examples of the measurement target component that increases over time include ammonia, potassium, Lactate Dehydrogenase (LDH), and free fatty acid. Examples of the measurement target component that decreases over time include amyloid β, blood glucose, free cholesterol, and triglycerides. In the following description, the component increase/decrease item is also referred to as a time increase/decrease item. Information about the component increase/decrease item includes, for example, a flag, a first threshold, information about a mode, a calculation formula, and an environmental condition.

The flag is information indicating that the measurement item is the component increase/decrease item. The first threshold is information about the time until an increase or decrease of the measurement target component affects a measurement result.

The information about the mode is information about a method (mode) for measurement of the component increase/decrease item in a case where the elapsed time has exceeded the first threshold. Examples of the mode for the measurement of the component increase/decrease item include a measurement execution mode for executing the measurement of the component increase/decrease item even in the case where the elapsed time has exceeded the first threshold, a measurement determination mode for allowing the user to determine whether to execute the measurement of the component increase/decrease item in the case where the elapsed time has exceeded the first threshold, and a measurement cancelation mode for canceling the measurement of the component increase/decrease item in the case where the elapsed time has exceeded the first threshold.

The calculation formula is a formula for calculating a component increase/decrease rate related to the ratio at which a measurement target component increases or decreases based on the elapsed time. The environmental condition is a condition for an environment in which the sample is stored. Examples of the condition in which the sample is stored include a cool and dark place, a room temperature, and refrigeration.

The information about the component increase/decrease item may not include all of the flag, the first threshold, the information about the mode, the calculation formula, and the environmental condition, and may include at least the flag and the first threshold. The information about the component increase/decrease item may also include information other than the flag, the first threshold, the information about the mode, the calculation formula, and the environmental condition.

The control circuitry 9 is a processor that functions as a core of the automatic analysis apparatus 1. The control circuitry 9 executes an operation program stored in the storage circuitry 8, thereby implementing a function corresponding to the operation program. The control circuitry 9 may include a storage area for storing at least part of data stored in the storage circuitry 8.

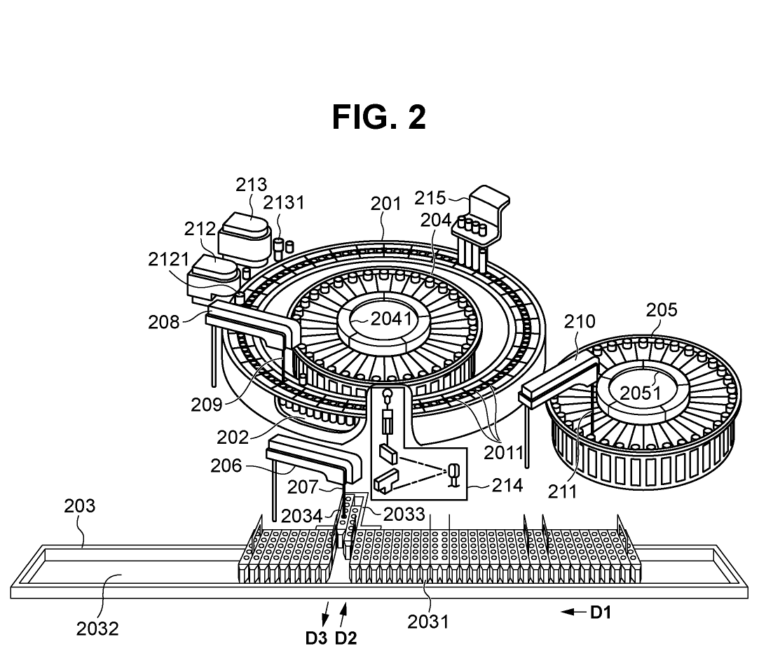

FIG. 2 is a schematic view illustrating a configuration of the analysis mechanism 2 according to the first embodiment. As illustrated in FIG. 2, the analysis mechanism 2 according to the first embodiment includes, for example, a reaction disk 201, a constant-temperature unit 202, a rack sampler 203, a first reagent storage 204, a second reagent storage 205, a sample dispensing arm 206, a sample dispensing probe 207, a first reagent dispensing arm 208, a first reagent dispensing probe 209, a second reagent dispensing arm 210, a second reagent dispensing probe 211, a first stirring unit 212, a second stirring unit 213, a photometry unit 214, and a cleaning unit 215.

The reaction disk 201 supports a plurality of reaction containers 2011 in such a manner that the reaction containers 2011 are arranged at a predetermined interval in an annular shape. The reaction disk 201 conveys the plurality of reaction containers 2011 along a predetermined path. Specifically, during an operation of analyzing a reaction solution of a sample and a reagent, the reaction disk 201 is alternately rotated and stopped at a predetermined time interval by the drive mechanism 4. Each reaction container 2011 is formed of, for example, glass, polypropylene (PP), or acrylic resin.

The constant-temperature unit 202 stores a heating medium set to a predetermined temperature. The constant-temperature unit 202 increases the temperature of the reaction solution contained in the reaction containers 2011 to the predetermined temperature and maintains its temperature by immersing the reaction containers 2011 in the stored heating medium.

The rack sampler 203 movably supports sample racks 2031, each capable of holding a plurality of sample containers. The plurality of sample containers each contains a blood sample that is a sample for which measurement has been requested. In an example illustrated in FIG. 2, each sample rack 2031 is capable of holding five sample containers that are aligned.

The rack sampler 203 includes a reader (not illustrated). The reader is provided, for example, at a position where an optical mark attached to each sample container can be read. The optical mark is a mark obtained by encoding identification information or the like about the sample contained in each sample container. Examples of the optical mark include a barcode, a one-dimensional pixel code, and a two-dimensional pixel code. The reader starts reading the optical mark triggered by an instruction to start ID reading from the control circuitry 9. The reader reads the identification information of the sample from the optical mark when the sample container arrives at a position where the optical mark can be read. The reader supplies the read identification information to the control circuitry 9. The reader may be replaced by another sensor using radio frequency identification (RFID) or the like.

The rack sampler 203 is provided with a conveyance area 2032 in which each sample rack 2031 is conveyed. Specifically, the conveyance area 2032 is used to convey each sample rack 2031 from an input position where the sample rack 2031 is input to a collection position where the sample rack 2031 is collected after the measurement is completed. In the conveyance area 2032, the plurality of sample racks 2031 aligned in a longitudinal direction is moved in a direction D1 by the drive mechanism 4.

The rack sampler 203 is provided with a pull-in area 2033 where each sample rack 2031 is pulled in from the conveyance area 2032 so as to move the sample container held by the sample rack 2031 toward a predetermined sample aspiration position. The sample aspiration position is set for example, at a position where a rotational trajectory of the sample dispensing probe 207 and a movement trajectory of an opening of the sample container supported by the rack sampler 203 and held by the sample rack 2031 intersect with each other. In the pull-in area 2033, the conveyed sample rack 2031 is moved in a direction D2 by the drive mechanism 4.

The rack sampler 203 is provided with a return area 2034 for returning the sample rack 2031 that holds the sample container from which the sample has been aspirated to the conveyance area 2032. In the return area 2034, the sample rack 2031 is moved in a direction D3 by the drive mechanism 4.

The mechanism for conveying sample containers is not limited to the rack sampler 203. In place of the rack sampler 203, a sample disk configured to hold one or more sample containers and to be rotated to convey the one or more sample containers to a predetermined position may be used.

The first reagent storage 204 refrigerates a plurality of reagent containers each containing a first reagent that reacts with a predetermined component contained in the standard sample and the test sample. The first reagent is, for example, a buffer solution containing bovine serum albumin (BSA) or the like. A reagent label is attached to each reagent container. An optical mark representing reagent information is printed on the reagent label. As the optical mark, for example, any pixel code such as a one-dimensional pixel code or a two-dimensional pixel code may be used.

The first reagent storage 204 refrigerates a plurality of standard sample containers each containing a standard sample. Each of the plurality of standard sample containers contains a standard sample of the same component at different concentrations.

In the first reagent storage 204, a reagent rack 2041 is rotatably provided. The reagent rack 2041 holds a plurality of reagent containers and a plurality of standard sample containers arranged in an annular shape. The reagent rack 2041 is rotated by the drive mechanism 4. In the first reagent storage 204, a reader (not illustrated) for reading the reagent information from the reagent label attached to each reagent container is provided. The read reagent information is stored in the storage circuitry 8.

At a predetermined position on the first reagent storage 204, a first reagent aspiration position is set. The first reagent aspiration position is set, for example, at a position where a rotational trajectory of the first reagent dispensing probe 209 and a movement trajectory of an opening of each of the reagent containers and the standard sample containers arranged in an annular shape in the reagent rack 2041 intersect with each other.

The second reagent storage 205 refrigerates a plurality of reagent containers each containing a second reagent paired with the first reagent in a two-reagent system. The second reagent is a solution containing an insoluble carrier, such as a carrier particle, on which an antigen or antibody that binds or dissociates through a specific antigen-antibody reaction with a predetermined antigen or antibody contained in the sample is immobilized. An entity that binds or dissociates through a specific reaction may be an enzyme, a substrate, an aptamer, or a receptor. In the second reagent storage 205, a reagent rack 2051 is rotatably provided. In the following description, in a case where the first reagent storage 204 and the second reagent storage 205 are expressed without distinction, these reagent storages may be simply referred to as a "reagent storage".

The reagent rack 2051 holds a plurality of reagent containers arranged in an annular shape. In the second reagent storage 205, the standard sample containers each containing the standard sample may be refrigerated. The reagent rack 2051 is rotated by the drive mechanism 4. In the second reagent storage 205, a reader (not illustrated) for reading reagent information from a reagent label attached to each reagent container is provided. The read reagent information is stored in the storage circuitry 8.

A second reagent aspiration position is set at a predetermined position on the second reagent storage 205. The second reagent aspiration position is set, for example, at a position where a rotational trajectory of the second reagent dispensing probe 211 and a movement trajectory of an opening of each of the reagent containers arranged in an annular shape in the reagent rack 2051 intersect with each other.

The sample dispensing arm 206 is provided between the reaction disk 201 and the rack sampler 203. The sample dispensing arm 206 is provided so as to be movable up and down in a vertical direction and rotatable in a horizontal direction by the drive mechanism 4. The sample dispensing arm 206 holds the sample dispensing probe 207 at one end thereof.

The sample dispensing probe 207 rotates along an arc-shaped rotational trajectory as the sample dispensing arm 206 rotates. On this rotational trajectory, a dispensing position for aspirating the sample from the sample container held by the rack sampler 203 is set. On the rotational trajectory of the sample dispensing probe 207, a sample discharge position for discharging the sample aspirated by the sample dispensing probe 207 to the reaction containers 2011 is set. The sample discharge position is set at a position where the rotational trajectory of the sample dispensing probe 207 and the movement trajectory of each reaction container 2011 held by the reaction disk 201 intersect with each other.

The sample dispensing probe 207 is driven by the drive mechanism 4 and moves in the up-down direction at the dispensing position or the sample discharge position. The sample dispensing probe 207 aspirates the sample from a sample container held by the rack sampler 203 at the dispensing position under the control of the control circuitry 9. Furthermore, the sample dispensing probe 207 discharges the aspirated sample to a reaction container 2011 located immediately below the sample discharge position under the control of the control circuitry 9.

The first reagent dispensing arm 208 is provided in the vicinity of the outer periphery of the first reagent storage 204. The first reagent dispensing arm 208 is provided so as to be movable up and down in the vertical direction and rotatable in the horizontal direction by the drive mechanism 4. The first reagent dispensing arm 208 holds the first reagent dispensing probe 209 at one end thereof.

The first reagent dispensing probe 209 rotates along an arc-shaped rotational trajectory as the first reagent dispensing arm 208 rotates. On this rotational trajectory, the above-described first reagent aspiration position is set. A first reagent discharge position where the first reagent or the standard sample aspirated by the first reagent dispensing probe 209 is discharged to the reaction containers 2011 is set on the rotational trajectory of the first reagent dispensing probe 209. The first reagent discharge position is set at a position where the rotational trajectory of the first reagent dispensing probe 209 and the movement trajectory of each reaction container 2011 held by the reaction disk 201 intersect with each other.

The first reagent dispensing probe 209 is driven by the drive mechanism 4 and moves in the up-down direction at the first reagent aspiration position or the first reagent discharge position on the rotational trajectory. The first reagent dispensing probe 209 aspirates the first reagent or the standard sample from a reagent container located immediately below the first reagent aspiration position under the control of the control circuitry 9. The first reagent dispensing probe 209 discharges the aspirated first reagent or standard sample to a reaction container 2011 located immediately below the first reagent discharge position under the control of the control circuitry 9.

The second reagent dispensing arm 210 is provided in the vicinity of the outer periphery of the second reagent storage 205. The second reagent dispensing arm 210 is provided so as to be movable up and down in the vertical direction and rotatable in the horizontal direction by the drive mechanism 4. The second reagent dispensing arm 210 holds the second reagent dispensing probe 211 at one end thereof.

The second reagent dispensing probe 211 rotates along an arc-shaped rotational trajectory as the second reagent dispensing arm 210 rotates. On this rotational trajectory, the above-described second reagent aspiration position is set. A second reagent discharge position where the second reagent aspirated by the second reagent dispensing probe 211 is discharged to the reaction containers 2011 is set on the rotational trajectory of the second reagent dispensing probe 211. The second reagent discharge position is set at a position where the rotational trajectory of the second reagent dispensing probe 211 and the movement trajectory of each reaction container 2011 held by the reaction disk 201 intersect with each other.

The second reagent dispensing probe 211 is driven by the drive mechanism 4 and moves in the up-down direction at the second reagent aspiration position or the second reagent discharge position on the rotational trajectory. The second reagent dispensing probe 211 aspirates the second reagent from a reagent container located immediately below the second reagent aspiration position under the control of the control circuitry 9. The second reagent dispensing probe 211 discharges the aspirated second reagent to a reaction container 2011 located immediately below the second reagent discharge position under the control of the control circuitry 9.

The first stirring unit 212 is provided in the vicinity of the outer periphery of the reaction disk 201. The first stirring unit 212 includes a first stirring arm 2121 and also includes a first stirrer provided at a distal end of the first stirring arm 2121. The first stirring unit 212 uses the first stirrer to stir the reaction solution of the standard sample and the first reagent contained in the reaction container 2011 located at a first stirring position on the reaction disk 201. Furthermore, the first stirring unit 212 uses the first stirrer to stir the reaction solution of the test sample and the first reagent contained in the reaction container 2011 located at the first stirring position on the reaction disk 201.

The second stirring unit 213 is provided in the vicinity of the outer periphery of the reaction disk 201. The second stirring unit 213 includes a second stirring arm 2131 and also includes a second stirrer provided at a distal end of the second stirring arm 2131. The second stirring unit 213 uses the second stirrer to stir the reaction solution of the standard sample, the first reagent, and the second reagent contained in the reaction container 2011 located at a second stirring position on the reaction disk 201. Furthermore, the second stirring unit 213 uses the second stirrer to stir the reaction solution of the test sample, the first reagent, and the second reagent contained in the reaction container 2011 located at the second stirring position.

The photometry unit 214 optically measures the reaction solution of the sample, the first reagent, and the second reagent discharged into the reaction container 2011. The photometry unit 214 includes a light source 2141 and a photodetector 2142. The photometry unit 214 emits light from the light source 2141 under the control of the control circuitry 9. The emitted light enters from a first side wall of the reaction container 2011 and exits from a second side wall opposite the first side wall. The photometry unit 214 uses the photodetector 2142 to detect the light exiting through the reaction container 2011.

Specifically, the photodetector 2142 is located, for example, at a position on an optical axis of the light emitted to the reaction containers 2011 from the light source 2141. The photodetector 2142 detects light that has passed through the reaction solution of the standard sample, the first reagent, and the second reagent in each reaction container 2011. The automatic analysis apparatus 1 acquires photometry data represented by intensity of light detected by the photodetector 2142. Then, the automatic analysis apparatus 1 generates standard data represented by absorbance based on measurement data acquired at a predetermined timing from the photometry data. Furthermore, the photodetector 2142 detects light that has passed through the reaction solution of the test sample, the first reagent, and the second reagent in the reaction container 2011. The automatic analysis apparatus 1 acquires photometry data represented by the intensity of light detected by the photodetector 2142. Then, the automatic analysis apparatus 1 generates test data represented by the absorbance based on the measurement data acquired at a predetermined timing from the photometry data. The photometry unit 214 outputs the generated standard data and test data to the analysis circuitry 3.

The cleaning unit 215 cleans the inside of each reaction container 2011 for which the measurement of the reaction solution is completed by the photometry unit 214.

The control circuitry 9 illustrated in FIG. 1 executes control programs stored in the storage circuitry 8, thereby implementing the functions corresponding to the programs. For example, the control circuitry 9 executes control programs to thereby implement a system control function 91, a flag setting function 92, a threshold setting function 93, an acquisition function 94, a calculation function 95, a formula setting function 96, a determination function 97, a mode setting function 98, a display control function 99, an output function 910, and a notification function 911. While, in the first embodiment, a case is described where the system control function 91, the flag setting function 92, the threshold setting function 93, the acquisition function 94, the calculation function 95, the formula setting function 96, the determination function 97, the mode setting function 98, the display control function 99, the output function 910, and the notification function 911 are implemented by a single processor, the first embodiment is not limited thereto. For example, a plurality of independent processors may be combined to constitute control circuitry, and the processors may execute control programs to thereby implement various functions.

The system control function 91 is a function for controlling each unit in the automatic analysis apparatus 1 in an integrated manner based on input information input from the input interface 5. For example, the control circuitry 9 uses the system control function 91 to control the drive mechanism 4 and the analysis mechanism 2, thereby controlling the sample dispensing arm 206, the first reagent dispensing arm 208, and the second reagent dispensing arm 210 to dispense a sample, a reagent, or the like into a reaction container while controlling the analysis circuitry 3 to execute an analysis according to each measurement item. The system control function 91 constitutes a system control unit according to the first embodiment.

The flag setting function 92 is a function for setting a flag on the component increase/decrease item. The flag setting function 92 constitutes a flag setting unit according to the first embodiment. The threshold setting function 93 is a function for setting the first threshold for the component increase/decrease item for which the flag is set. The threshold setting function 93 constitutes a threshold setting unit according to the first embodiment.

The acquisition function 94 is a function for acquiring time information. The time information is information including at least one of a collection time, a measurement request time, and an initial measurement time. The collection time is a time when a sample is collected. The collection time is, for example, a blood collection time that is a time when a blood sample is collected. The measurement request time is, for example, a time when examination order information is created, or a time when the examination order information is transmitted. The initial measurement time is a time when a first measurement of a measurement item is carried out. The time information is stored in, for example, the storage circuitry 8. The acquisition function 94 constitutes an acquisition unit according to the first embodiment.

The calculation function 95 is a function for calculating an elapsed time from the time included in the time information to the current time. The calculation function 95 constitutes a calculation unit according to the first embodiment. The formula setting function 96 is a function for setting a formula based on the elapsed time calculated by the calculation function 95 on the component increase/decrease item for which the flag is set. The formula setting function 96 is also a function for setting an environmental condition on the formula. The formula setting function 96 constitutes a formula setting unit according to the first embodiment.

The determination function 97 is a function for determining whether the elapsed time calculated by the calculation function 95 has exceeded the first threshold. The determination function 97 constitutes a determination unit according to the first embodiment. The mode setting function 98 is a function for setting a mode for executing measurement of the component increase/decrease item in a case where the elapsed time has exceeded the first threshold. Specifically, the mode setting function 98 sets any of the measurement execution mode, the measurement determination mode, and the measurement cancelation mode as the mode for executing the measurement of the component increase/decrease item on the component increase/decrease item for which the flag is set. The mode setting function 98 constitutes a mode setting unit according to the first embodiment.

The display control function 99 is a function for controlling the display circuitry of the output interface 6 to display a measurement availability selection screen for allowing the user to select whether measurement is possible in a case where the measurement determination mode is set on the component increase/decrease item and the elapsed time has exceeded the first threshold. The display control function 99 is a function for controlling the display circuitry of the output interface 6 to display an item setting screen to be described below. The item setting screen includes at least the flag and the first threshold. The item setting screen may further include the time information in addition to the flag and the first threshold. The item setting screen may further include a determination result that indicates whether the elapsed time has exceeded the first threshold and determined by the determination function 97, in addition to the flag, the first threshold, and the time information. The item setting screen corresponds to a setting screen according to the first embodiment. The display control function 99 constitutes a first display control unit and a second display control unit according to the first embodiment.

The output function 910 is a function for outputting at least one of the elapsed time and the component increase/decrease rate calculated by the calculation function 95 in association with a measurement result for a measurement target component in the case where the elapsed time has exceeded the first threshold. The output function 910 constitutes an output unit according to the first embodiment. The notification function 911 is a function for notifying the user of cancellation of measurement of the component increase/decrease item in a case where the measurement cancelation mode is set for the component increase/decrease item and the elapsed time has exceeded the first threshold. The notification function 911 constitutes a notification unit according to the first embodiment.

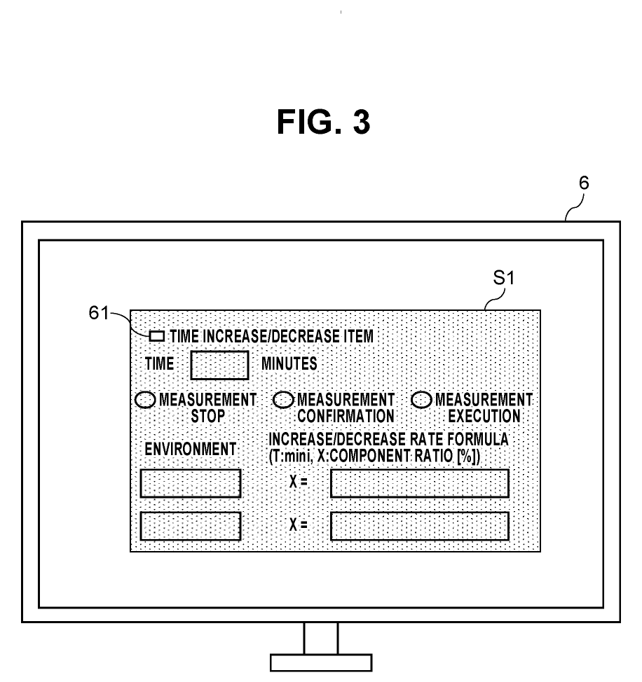

An example of settings for various kinds of information included in the information about the component increase/decrease item will be described with reference to FIGS. 3 to 6. FIGS. 3 to 6 each illustrate an example of the item setting screen. An item setting screen S1 is, for example, a screen included in an item parameter screen for setting various parameters included in the measurement item. In a case where a measurement target component of the measurement item on the item parameter screen is a measurement target component that increases or decreases over time, the information about the component increase/decrease item is set by the user on the item setting screen S1. The item setting screen S1 is displayed on the display circuitry of the output interface 6 by the display control function 99 or the output function 910.

As illustrated in FIG. 3, a checkbox 61 for setting a flag indicating a time increase/decrease item (component increase/decrease item) is displayed on the item setting screen S1 displayed on the display circuitry of the output interface 6 in a form that allows input by the user. In a case where the measurement target component of the measurement item on the item parameter screen is a measurement target component that increases or decreases over time, the user checks the checkbox 61 via the input interface 5. Accordingly, the flag setting function 92 sets the flag on the time increase/decrease item based on an input operation of checking the checkbox 61 by the user.

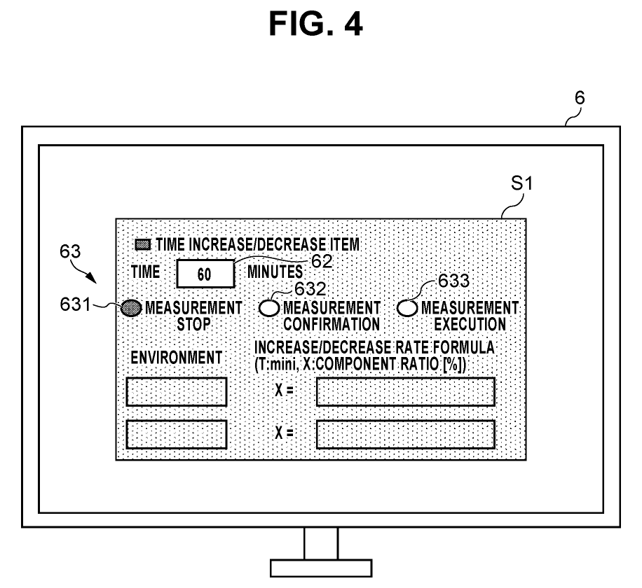

As illustrated in FIG. 4, by the checkbox 61 being checked on the item setting screen S1, a first threshold input box 62 for inputting the first threshold relating to the time until an increase or decrease of the measurement target component affects the measurement result is displayed in a form that allows input by the user. The user inputs a numerical value to the first threshold input box 62 via the input interface 5. Accordingly, the threshold setting function 93 sets the first threshold for the time increase/decrease item for which the flag is set based on an input operation of inputting the numerical value to the first threshold input box 62. In the example illustrated in FIG. 4, "60" is input to the first threshold input box 62, and thus the threshold setting function 93 sets "60 minutes" as the first threshold.

As illustrated in FIG. 4, by the checkbox 61 being checked on the item setting screen S1, a mode input box 63 for setting information about the mode is displayed in a form that allows input by the user. In the example illustrated in FIG. 4, the mode input box 63 on the item setting screen S1 includes a measurement stop input box 631 for setting the measurement cancelation mode, a measurement confirmation input box 632 for setting the measurement determination mode, and a measurement execution input box 633 for setting the measurement execution mode. The user checks any of the measurement stop input box 631, the measurement confirmation input box 632, and the measurement execution input box 633 via the input interface 5. Accordingly, the mode setting function 98 sets the mode for the time increase/decrease item for which the flag is set based on an input operation of checking any of the measurement stop input box 631, the measurement confirmation input box 632, and the measurement execution input box 633. In the example illustrated in FIG. 4, the measurement stop input box 631 is checked, and thus the mode setting function 98 sets the measurement cancelation mode.

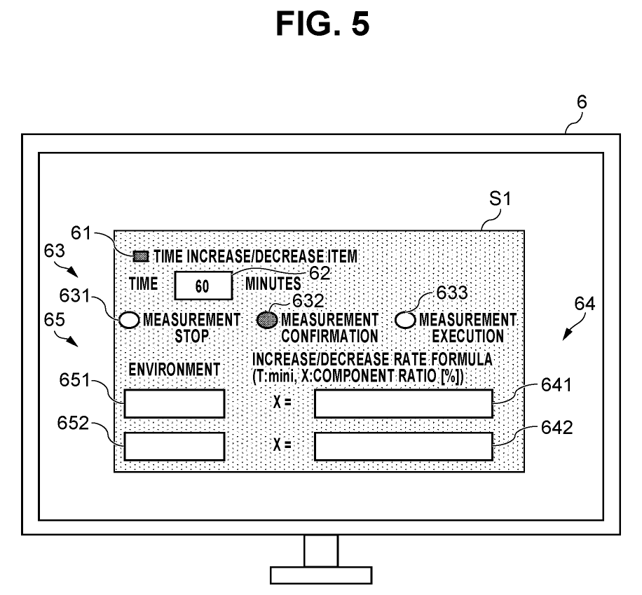

As illustrated in FIG. 5, by the measurement confirmation input box 632 being checked on the item setting screen S1, i.e., by the mode setting function 98 setting the measurement determination mode for the time increase/decrease item for which the flag is set, a formula input box 64 for inputting an increase/decrease rate formula (calculation formula) and an environment input box 65 for inputting an environmental condition are displayed in a form that allows input by the user. In the example illustrated in FIG. 5, two formula input boxes 64 and two environment input boxes 65 are displayed. In a case where the user inputs a formula to the formula input box 64 via the input interface 5, the formula setting function 96 sets the formula for the time increase/decrease item for which the flag is set based on an input operation of inputting the formula to the formula input box 64. In a case where the user inputs an environmental condition to the environment input box 65 via the input interface 5, the formula setting function 96 sets the environmental condition for the formula based on an input operation of inputting the environmental condition to the environment input box 65. In the example illustrated in FIG. 5, while two formula input boxes 64 and two environment input boxes 65 are displayed, the number of formula input boxes 64 and the number of environment input boxes 65 to be displayed on the item setting screen S1 are not limited to two. In other words, any number of formula input boxes 64 and any number of environment input boxes 65 may be displayed on the item setting screen S1. One or three or more formula input boxes 64 and environment input boxes 65 may be displayed.

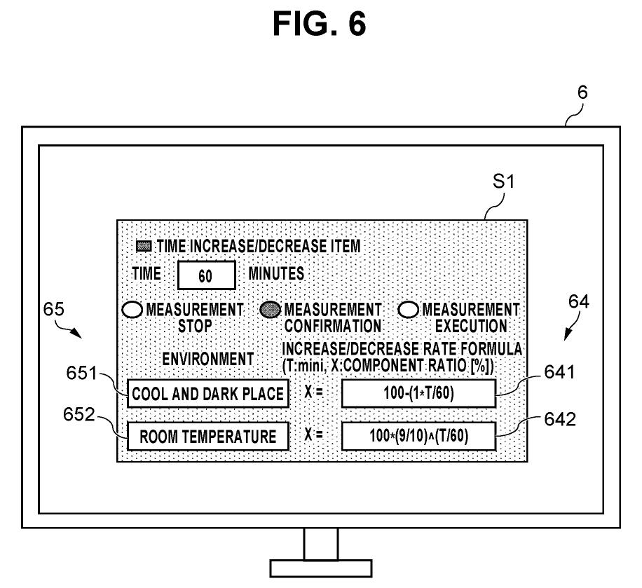

In the example illustrated in FIG. 6, "100 - (1*T/60)" is input to a first formula input box 641 in the formula input box 64, and thus "cool and dark place" is input to a first environment input box 651 in the environment input box 65 corresponding to the first formula input box 641. Specifically, the formula setting function 96 sets "X = 100 - (1*T/60)" as the formula and sets "cool and dark place" as the environmental condition for "X = 100 - (1*T/60)". X represents a component ratio [%] indicating a component increase/decrease rate. T represents an elapsed time [min]. In the example illustrated in FIG. 6, "100*(9/10)Ʌ(T/60)" is input to a second formula input box 642 in the formula input box 64, and "room temperature" is input to a second environment input box 652 in the environment input box 65 corresponding to the second formula input box 642. Specifically, the formula setting function 96 sets "X = 100*(9/10)Ʌ(T/60)" as the formula and sets "room temperature" as the environmental condition for "X = 100*(9/10)Ʌ(T/60)". In the example illustrated in FIG. 6, when the sample is stored in a cool and dark place, the measurement target components decrease by 1% per hour (60 minutes) only, and when the sample is stored at a room temperature, the measurement target components decrease by 10% per hour (60 minutes) only.

In the examples illustrated in FIGS. 5 and 6, by the measurement confirmation input box 632 being checked, or by the mode setting function 98 setting the measurement determination mode for the time increase/decrease item for which the flag is set, the formula input box 64 for inputting an increase/decrease rate formula (formula) and the environment input box 65 for inputting an environmental condition are each displayed in a form that allows input by the user. However, by the measurement execution input box 633 being checked, or by the mode setting function 98 setting the measurement execution mode for the time increase/decrease item for which the flag is set, the formula input box 64 for inputting the increase/decrease rate formula (formula) and the environment input box 65 for inputting an environmental condition are displayed in a form that allows input by the user, as in the case of setting the measurement determination mode.

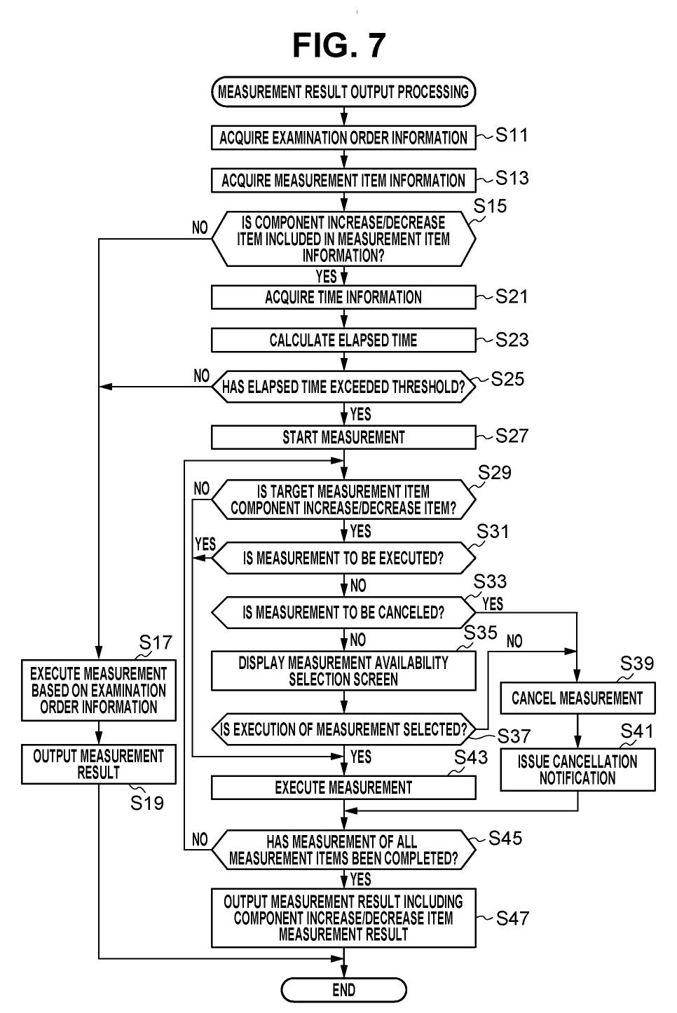

FIG. 7 is a flowchart illustrating contents of measurement result output processing executed by the automatic analysis apparatus 1 according to the first embodiment. In this measurement result output processing, a measurement is executed, and a measurement result is output. For example, the measurement result output processing is processing to be executed at a timing when an optical mark attached to each sample container is read.

As illustrated in FIG. 7, in step S11, first, the acquisition function 94 of the control circuitry 9 acquires examination order information. Specifically, the acquisition function 94 acquires, from the storage circuitry 8, examination order information about the sample contained in the sample container from which the optical mark is read. The acquisition function 94 may acquire examination order information from an external apparatus via the communication interface 7.

Next, as illustrated in FIG. 7, in step S13, the acquisition function 94 of the control circuitry 9 acquires measurement item information. Specifically, the acquisition function 94 acquires the measurement item information from the storage circuitry 8 based on the examination order information acquired in step S11.

Next, as illustrated in FIG. 7, in step S15, the determination function 97 of the control circuitry 9 determines whether the component increase/decrease item is included in the measurement item information. Specifically, the determination function 97 determines whether the flag indicating the component increase/decrease item is set in the measurement item information acquired in step S13, thereby determining whether the component increase/decrease item is included in the measurement item information.

In step S15, if the component increase/decrease item is not included in the measurement item information (NO in step S15), the processing proceeds to step S17. In step S17, the system control function 91 of the control circuitry 9 executes measurement based on the examination order information. Specifically, the system control function 91 executes the measurement based on the examination order information by executing the measurement item included in the examination order information based on the measurement item information acquired in step S13.

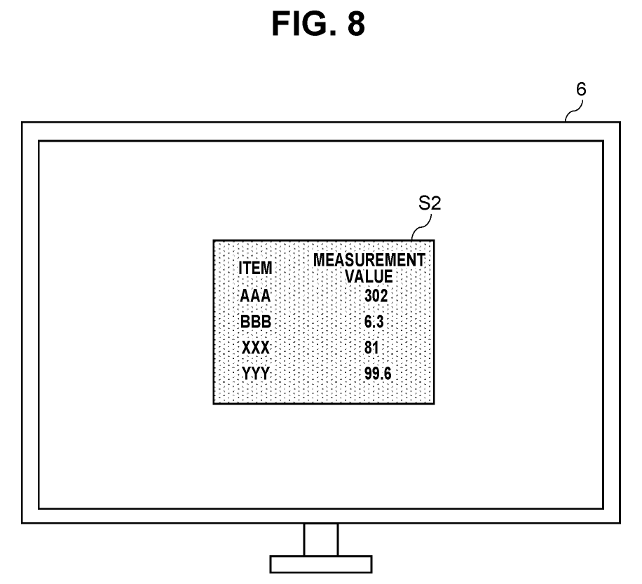

Next, as illustrated in FIG. 7, in step S19, the output function 910 of the control circuitry 9 outputs the measurement result. Specifically, the output function 910 displays the result of the measurement executed in step S17 on the display circuitry of the output interface 6, thereby outputting the measurement result. FIG. 8 illustrates an example of a measurement result screen. As illustrated in FIG. 8, the output function 910 outputs the measurement result screen including the measurement result, in which item names of four measurement items are associated with respective measurement values corresponding to the four measurement items, to the display circuitry of the output interface 6.

On the other hand, in step S15, if the component increase/decrease item is included in the measurement item information (YES in step S15), the processing proceeds to step S21. In step S21, the acquisition function 94 of the control circuitry 9 acquires time information. Specifically, the acquisition function 94 acquires time information from the storage circuitry 8. In the first embodiment, the acquisition function 94 acquires the blood collection time, which is an example of the collection time, as the time information. The acquisition function 94 may acquire the blood collection time, which is an example of the collection time, from an external apparatus via the communication interface 7.

Next, as illustrated in FIG. 7, in step S23, the calculation function 95 of the control circuitry 9 calculates an elapsed time. Specifically, the calculation function 95 calculates the elapsed time to a current time based on the time information acquired in step S21. More specifically, since the blood collection time is acquired in step S21, the calculation function 95 according to the first embodiment calculates the elapsed time from the blood collection time to the current time.

Next, as illustrated in FIG. 7, in step S25, the determination function 97 of the control circuitry 9 determines whether the elapsed time has exceeded a threshold. Specifically, the determination function 97 determines whether the elapsed time calculated in step S25 has exceeded the first threshold set for the component increase/decrease item for which the flag is set in the measurement item information acquired in step S13, thereby determining whether the elapsed time has exceeded the threshold. Then, if the elapsed time has not exceeded the threshold (NO in step S25), the processing proceeds to step S17. Then, the processing of steps S17 and S19 described above is executed.

On the other hand, if the elapsed time has exceeded the threshold (YES in step S25), the processing proceeds to step S27. In step S27, the system control function 91 of the control circuitry 9 starts measurement. Specifically, the system control function 91 starts the measurement based on the examination order information acquired in step S11.

Next, as illustrated in FIG. 7, in step S29, the system control function 91 of the control circuitry 9 determines whether the target measurement item corresponds to the component increase/decrease item. Specifically, the system control function 91 determines whether the target measurement item among measurement items included in the examination order information acquired in step S11 corresponds to the component increase/decrease item for which the flag is set.

If the target measurement item corresponds to the component increase/decrease item (YES in step S29), the processing proceeds to step S31. In step S31, the system control function 91 of the control circuitry 9 determines whether the measurement is to be executed. Specifically, the system control function 91 determines whether the measurement is to be executed based on information about the mode set for the component increase/decrease item for which the flag is set. More specifically, the system control function 91 determines whether the measurement execution mode is set, thereby determining whether the measurement is to be executed.

In step S31, if the measurement is not to be executed (NO in step S31), the processing proceeds to step S33. In step S33, the system control function 91 of the control circuitry 9 determines whether the measurement is to be canceled. Specifically, the system control function 91 determines whether the measurement is to be canceled based on information about the mode set for the component increase/decrease item for which the flag is set. More specifically, the system control function 91 determines whether the measurement cancelation mode is set, thereby determining whether the measurement is to be canceled.

If the measurement is not to be canceled (NO in step S33), the processing proceeds to step S35. In step S35, the display control function 99 of the control circuitry 9 displays the measurement availability selection screen. Specifically, the display control function 99 displays the measurement availability selection screen for allowing the user to select whether to execute the measurement on the display circuitry of the output interface 6. In the first embodiment, determination of the measurement execution mode, the measurement determination mode, or the measurement cancelation mode is performed each time the component increase/decrease item is to be measured. Accordingly, the display control function 99 according to the first embodiment displays the measurement availability selection screen each time the component increase/decrease item for which the measurement determination mode is set is to be measured.

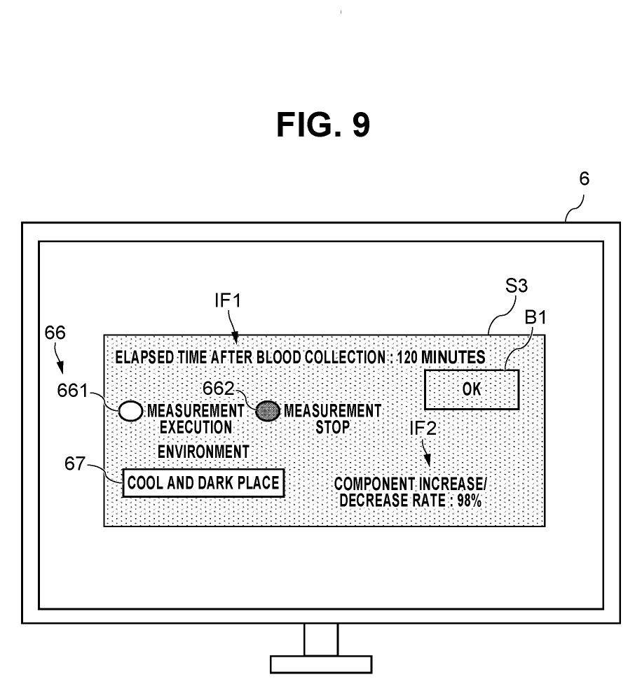

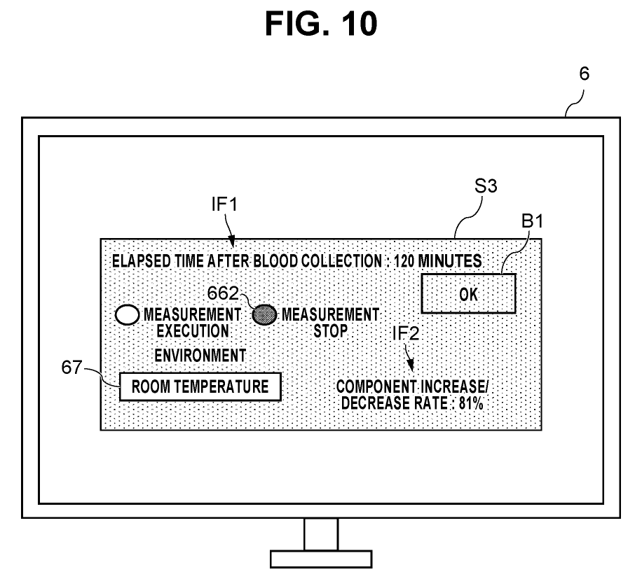

The measurement availability selection screen will be described with reference to FIGS. 9 and 10. FIG. 9 illustrates an example of the measurement availability selection screen according to the first embodiment. FIG. 10 illustrates another example of the measurement availability selection screen according to the first embodiment.

As illustrated in FIG. 9, a mode input box 66 for inputting a mode, an environmental information input box 67 for inputting environmental information, and an OK button B1 are displayed on a measurement availability selection screen S3. The environmental information is information about the environment in which the sample is stored. By the environmental information input box 67 being displayed on the measurement availability selection screen S3, environmental information can be input on the measurement availability selection screen S3. In the example illustrated in FIG. 9, the mode input box 66 includes a measurement execution input box 661 and a measurement stop input box 662, and the measurement stop input box 662 is checked. In the example illustrated in FIG. 9, "cool and dark place" is input to the environmental information input box 67.

As illustrated in FIG. 9, elapsed time information IF1 regarding the elapsed time and information IF2 that is information about the component increase/decrease rate in the environment are displayed. The elapsed time information IF1 indicates the elapsed time calculated in step S23. In the example illustrated in FIG. 9, "120 minutes" is displayed for the elapsed time after blood collection that is the elapsed time from the blood collection time to the current time. The information IF2 about the component increase/decrease rate includes the component increase/decrease rate calculated from the formula based on the elapsed time calculated in step S23 and the environmental information input to the environmental information input box 67. In the example illustrated in FIG. 9, the information IF2 about the component increase/decrease rate is displayed as "component increase/decrease rate: 98%" using the formula "X = 100 - (1*T/60)" set for the component increase/decrease item illustrated in FIG. 6 based on the elapsed time "120 minutes" and the environmental information "cool and dark place". On the other hand, in the example illustrated in FIG. 10, the information IF2 about the component increase/decrease rate is displayed as "component increase/decrease rate: 81%" using the formula "X = 100*(9/10)Ʌ(T/60)" set for the component increase/decrease item illustrated in FIG. 6 based on the elapsed time "120 minutes" and the environmental information "room temperature".

The user checks either the measurement execution input box 661 or the measurement stop input box 662 via the input interface 5 with reference to the elapsed time information IF1 and the component increase/decrease rate information IF2 on the measurement availability selection screen S3, and presses the OK button B1.

Next, as illustrated in FIG. 7, in step S37, the system control function 91 determines whether execution of measurement is selected. Specifically, the system control function 91 determines whether the execution of measurement is selected based on a user input operation on the measurement availability selection screen S3. More specifically, if the measurement execution input box 661 is checked on the measurement availability selection screen S3, the system control function 91 determines that the execution of measurement is selected. If the measurement stop input box 662 is checked, the system control function 91 determines that the execution of measurement is not selected, i.e., cancellation of measurement is selected.

If the measurement is to be canceled (YES in step S33), or if the execution of measurement is not selected (NO in step S37), the processing proceeds to step S39. In step S39, the system control function 91 of the control circuitry 9 cancels the measurement. Specifically, the system control function 91 skips the measurement of the component increase/decrease item to be measured, thereby canceling the measurement.

Next, as illustrated in FIG. 7, in step S41, the notification function 911 issues a cancellation notification. Specifically, the notification function 911 notifies the user of cancellation of the measurement of the component increase/decrease item in step S39. More specifically, the notification function 911 displays a notification screen including a message indicating that the measurement of the component increase/decrease item has been canceled on the display circuitry of the output interface 6, thereby issuing the cancellation notification. As a method for issuing the cancellation notification, the notification screen is displayed on the display circuitry of the output interface 6. However, the method for issuing the cancellation notification is not limited thereto. More specifically, any method for issuing cancellation notification may be employed. For example, the notification function 911 may issue the cancellation notification by printing the message indicating the cancellation on paper using the print circuitry of the output interface 6.

On the other hand, if the target measurement item does not correspond to the component increase/decrease item (NO in step S29), the processing proceeds to step S31. If the measurement is to be executed (YES in step S31) and execution of measurement is selected in step S37 (YES in step S37), the processing proceeds to step S43. In step S43, the system control function 91 of the control circuitry 9 executes the measurement. Specifically, the system control function 91 executes the measurement based on the measurement item information acquired in step S13.

Next, as illustrated in FIG. 7, after steps S41 and S43, the system control function 91 of the control circuitry 9 determines whether the measurement has all been completed. Specifically, in step S45, the system control function 91 determines whether the measurement of all measurement items included in the examination order information has been completed. If the measurement of all the measurement items has not been completed (NO in step S45), the processing returns to step S29. The processing from step S29 is repeatedly performed on the subsequent measurement item, and the control circuitry 9 waits until the measurement of all the measurement items is completed.

On the other hand, if the measurement of all the measurement items has been completed (YES in step S45), the processing proceeds to step S47. In step S47, the output function 910 of the control circuitry 9 outputs measurement results including the measurement result for the component increase/decrease item. Specifically, the output function 910 outputs the measurement result screen including the measurement result for the component increase/decrease item to the display circuitry of the output interface 6, thereby outputting the measurement results. As a method for outputting the measurement results, the measurement result screen is displayed on the display circuitry of the output interface 6. However, the method for outputting the measurement results is not limited thereto. More specifically, any method for outputting the measurement results may be employed. The output function 910 may output the measurement results by, for example, printing the measurement results on paper using the print circuitry of the output interface 6.

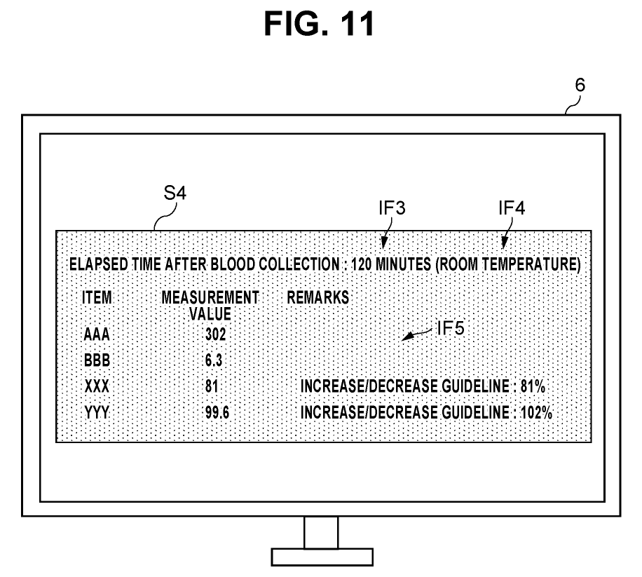

FIG. 11 illustrates an example of the measurement result screen including the measurement result for the component increase/decrease item according to the first embodiment. As illustrated in FIG. 11, elapsed time information IF3, environmental information IF4, and a measurement result IF5 are output on a measurement result screen S4 including the measurement result for the component increase/decrease item.

The elapsed time information IF3 included in the measurement result screen S4 indicates the elapsed time calculated in step S23. In the example illustrated in FIG. 11, "elapsed time after blood collection: 120 minutes" is displayed as the elapsed time information IF3. The environmental information IF4 included in the measurement result screen S4 is the environmental information input to the environmental information input box 67 on the measurement availability selection screen S3. In the example illustrated in FIG. 11, "(room temperature)" is displayed as the environmental information. The measurement result IF5 included in the measurement result screen S4 includes measurement items included in the examination order information, measurement values obtained by executing the measurement, and information about the component increase/decrease rate. In the example illustrated in FIG. 11, four measurement items, measurement values respectively corresponding to the four measurement items, and information about the component increase/decrease rate for two component increase/decrease items are displayed as a measurement result. These measurement values are actually measured values. In the example illustrated in FIG. 11, since a measurement item "XXX" and a measurement item "YYY" each correspond to the component increase/decrease item for which the flag is set, "increase/decrease guideline: 81%" is displayed as a guideline value for the component increase/decrease rate corresponding to the measurement item "XXX", and the "increase/decrease guideline: 102%" is displayed as a guideline value for the component increase/decrease rate corresponding to the measurement item "YYY" as information about the component increase/decrease rate in a remarks column of the measurement result screen S4. The user can calculate a correction amount of each measurement value with reference to the guideline value for the component increase/decrease rate.

On the measurement result screen S4 including the measurement result for the component increase/decrease item, both the elapsed time information IF3 and information about the component increase/decrease rate in the measurement result IF5 are output. However, on the measurement result screen S4 including the measurement result for the component increase/decrease item, at least one of the elapsed time information IF3 and the information about the component increase/decrease rate in the measurement result IF5 may be output.

After execution of step S19 or step S47, the measurement result output processing ends.

As described above, since the automatic analysis apparatus 1 according to the first embodiment has the flag setting function 92 and the threshold setting function 93, it is possible to set the flag for the component increase/decrease item and set the first threshold for the component increase/decrease item. Accordingly, it is possible to reduce the probability that an increase or decrease in the measurement target component over time affects a diagnosis.

In the automatic analysis apparatus 1 according to the first embodiment described above, in a case where the measurement determination mode is set and the examination order information includes a plurality of component increase/decrease items whose elapsed time has exceeded the threshold, it is possible to allow the user to select measurement availabilities for the plurality of component increase/decrease items individually for each component increase/decrease item on a single measurement availability selection screen, or to select the measurement availabilities for the plurality of component increase/decrease items collectively on the single measurement availability selection screen.

Further, in the automatic analysis apparatus 1 according to the first embodiment described above, the environmental information IF4 included in the measurement result screen S4 may be made selectable in a case where the measurement execution mode is set and the examination order information includes a plurality of component increase/decrease items whose elapsed times have exceeded the threshold. Furthermore in a case where the environmental information is switched by an input operation by the user, the output function 910 may recalculate the guideline value for the component increase/decrease rate, which is information about the component increase/decrease rate included in the measurement result screen S4, based on switched environmental information, and may output the calculated guideline value to the measurement result screen S4.

### Second Embodiment

In the automatic analysis apparatus 1 according to the first embodiment described above, as settings for various kinds of information included in the information about the component increase/decrease item, the flag, the first threshold, the information about the mode, the formula, and the environmental condition are all configured on the item setting screen. However, the settings for various kinds of information are not limited thereto. In a second embodiment, as settings for various kinds of information included in the information about the component increase/decrease item, the flag and the first threshold may be configured. A case where this modified example is applied to the first embodiment will be described as the second embodiment, and differences from the first embodiment will be described.

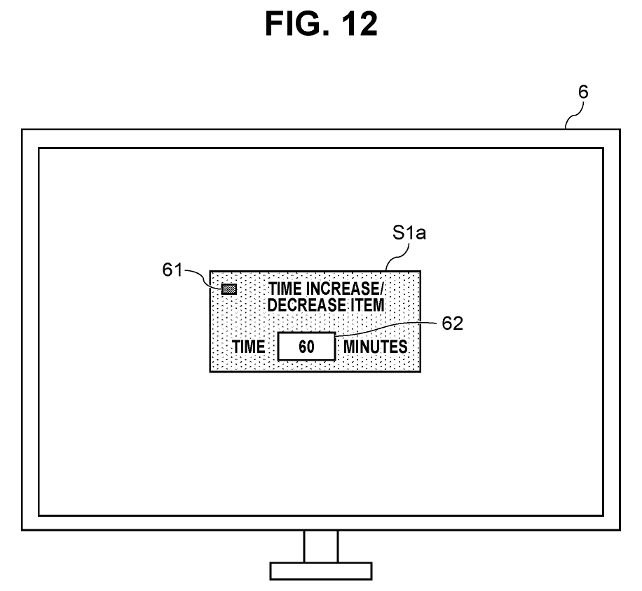

FIG. 12 illustrates an example of an item setting screen according to the second embodiment, and FIG. 12 corresponds to FIG. 6. As illustrated in FIG. 12, on an item setting screen S1a displayed on the display circuitry of the output interface 6, the checkbox 61 for setting the flag indicating the time increase/decrease item (component increase/decrease item) and the first threshold input box 62 for inputting the first threshold relating to the time until an increase or decrease of a measurement target component affects a measurement result are displayed in a form that allows input by the user. In the example illustrated in FIG. 12, the checkbox 61 is checked, and thus the flag setting function 92 sets the flag on the time increase/decrease item. In the example illustrated in FIG. 12, "60" is input to the first threshold input box 62, and thus the threshold setting function 93 sets "60 minutes" as the first threshold.

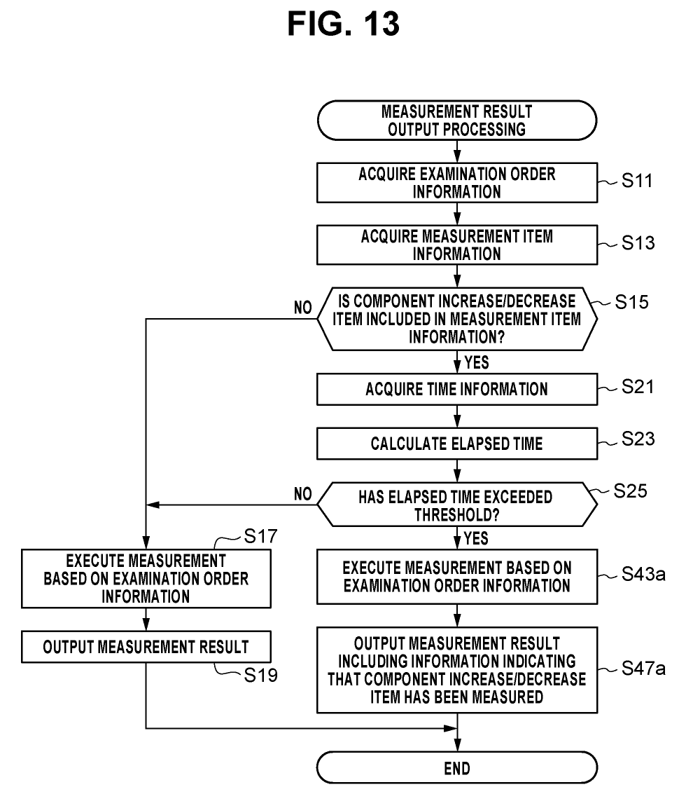

FIG. 13 is a flowchart illustrating contents of measurement result output processing executed by the automatic analysis apparatus 1 according to the second embodiment, and FIG. 13 corresponds to FIG. 7. In this measurement result output processing, a measurement is executed, and a measurement result is output. For example, the measurement result output processing is processing executed at a timing when sample information is read. The processing of steps S11 to S25 is similar to the processing of steps S11 to S25 illustrated in FIG. 7, and thus the descriptions thereof are omitted.

Next, as illustrated in FIG. 13, if the elapsed time has exceeded the threshold (YES in step S25), the processing proceeds to step S43a. In step S43a, the system control function 91 of the control circuitry 9 executes the measurement based on the examination order information. Specifically, the system control function 91 executes the measurement of a measurement item included in the examination order information based on the measurement item information acquired in step S13, thereby executing the measurement based on the examination order information. More specifically, in the second embodiment, the measurement of the measurement item included in the examination order information that includes the component increase/decrease item for which the flag is set is executed based on the measurement item information acquired in step S13.

Next, as illustrated in FIG. 13, in step S47a, the output function 910 of the control circuitry 9 outputs a measurement result including information indicating that the component increase/decrease item has been measured. Specifically, the output function 910 outputs the measurement result screen including the measurement result including the information indicating that the component increase/decrease item has been measured to the display circuitry of the output interface 6, thereby outputting the measurement result.

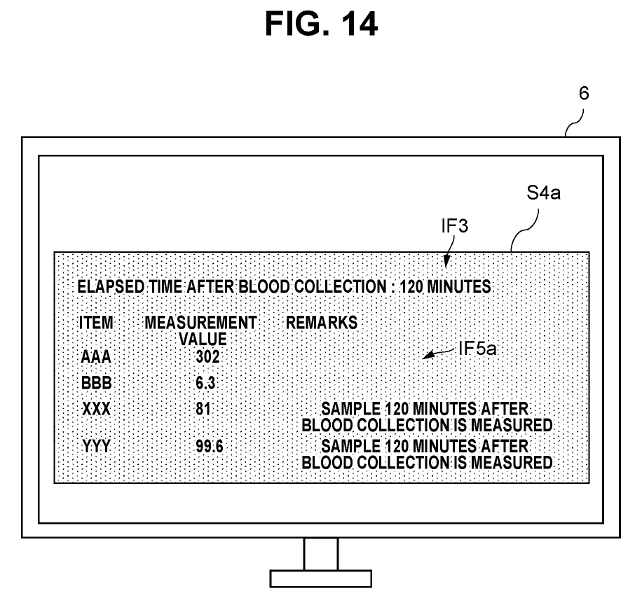

FIG. 14 illustrates an example of the measurement result screen including the measurement result including the information indicating that the component increase/decrease item has been measured according to the second embodiment, and FIG. 14 corresponds to FIG. 11. As illustrated in FIG. 14, on a measurement result screen S4a, the elapsed time information IF3 and a measurement result IF5a including the information indicating that the component increase/decrease item has been measured are output.

The elapsed time information IF3 included in the measurement result screen S4a indicates the elapsed time calculated in step S23. As in the example illustrated in FIG. 11, "elapsed time after blood collection: 120 minutes" is displayed as the elapsed time information IF3. The measurement result IF5a included in the measurement result screen S4a includes the measurement items included in the examination order information and the measurement values obtained by executing the measurement. In the example illustrated in FIG. 14, four measurement items, measurement values respectively corresponding to the four measurement items, and information indicating that the component increase/decrease item has been measured are displayed. Further, in the example illustrated in FIG. 14, the measurement result IF5a included in the measurement result screen S4a includes "the sample 120 minutes after blood collection is measured ", which is information indicating that the component increase/decrease item has been measured, in a remarks column for each of the measurement item "XXX" and the measurement item "YYY". The user confirms the information indicating that the component increase/decrease item has been measured that is output on the measurement result screen S4a, and can confirm that the component increase/decrease item is included in the measurement result.

As described above, since the automatic analysis apparatus 1 according to the first embodiment has the flag setting function 92 and the threshold setting function 93, it is possible to set the flag for the component increase/decrease item and set the first threshold for the component increase/decrease item. Accordingly, it is possible to reduce the probability that an increase or decrease of a measurement target component over time affects a diagnosis.

### First Modified Example

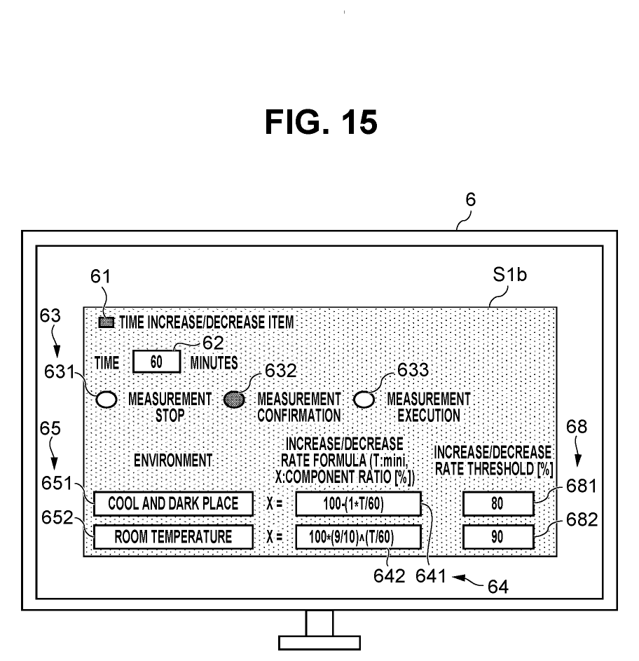

In the automatic analysis apparatus 1 according to the first embodiment described above, it is also possible to set a threshold for the component increase/decrease rate for the formula. FIG. 15 illustrates an example of an item setting screen according to a first modified example, and FIG. 15 corresponds to FIG. 6. As illustrated in FIG. 15, by the measurement confirmation input box 632 being checked on an item setting screen S1b, or by the mode setting function 98 setting the measurement determination mode for the time increase/decrease item for which the flag is set, the formula input box 64 for inputting the increase/decrease rate formula (formula), the environment input box 65 for inputting the environmental condition, and an increase/decrease rate threshold input box 68 for setting an increase/decrease rate threshold for the component increase/decrease rate are displayed on the item setting screen S1b in a form that allows input by the user. The formula input box 64 for inputting the increase/decrease rate formula (formula) and the environment input box 65 for inputting the environmental condition are similar to those of the first embodiment, and thus descriptions thereof are omitted.

In a case where the user inputs the increase/decrease rate threshold to the increase/decrease rate threshold input box 68 via the input interface 5, the threshold setting function 93 sets the increase/decrease rate threshold for the formula based on an input operation of inputting the increase/decrease rate threshold to the increase/decrease rate threshold input box 68. This increase/decrease rate threshold corresponds to a second threshold. In the example illustrated in FIG. 15, two input boxes, i.e., a first increase/decrease rate threshold input box 681 and a second increase/decrease rate threshold input box 682, are displayed as the increase/decrease rate threshold input box 68. In the example illustrated in FIG. 15, "80" is input to the first increase/decrease rate threshold input box 681 of the increase/decrease rate threshold input box 68. In other words, the threshold setting function 93 sets "80%" as the increase/decrease rate threshold. Furthermore, in the example illustrated in FIG. 15, "90" is input to the second increase/decrease rate threshold input box 682 of the increase/decrease rate threshold input box 68. In other words, the threshold setting function 93 sets "90%" as the increase/decrease rate threshold.

By setting the increase/decrease rate thresholds as described above, for example, in step S35 of the measurement result output processing illustrated in FIG. 7, the display control function 99 does not display the measurement availability selection screen, and the calculation function 95 calculates the component increase/decrease rate using the formula and compares the calculated component increase/decrease rate with the increase/decrease rate threshold. Thus, in step S37 of the measurement result output processing illustrated in FIG. 7, the system control function 91 determines that execution of measurement is selected in a case where the calculated component increase/decrease rate is greater than the increase/decrease rate threshold, and the system control function 91 determines that the execution of measurement is not selected, i.e. cancellation of measurement is selected, in a case where the calculated component increase/decrease rate is smaller than the increase/decrease rate threshold.

### Second Modified Example

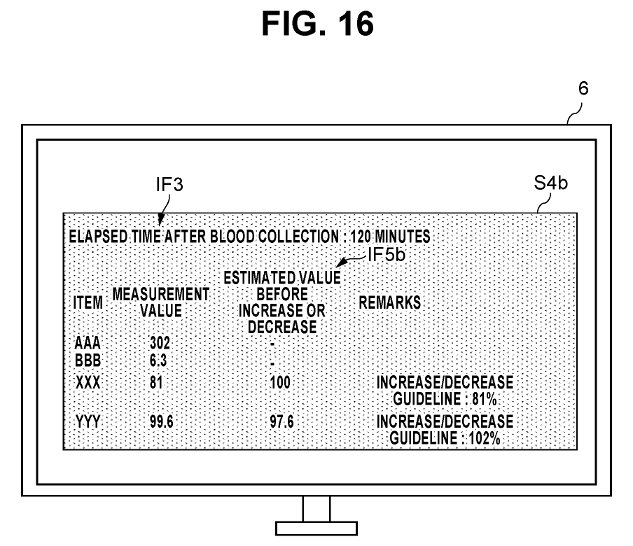

In the automatic analysis apparatus 1 according to the first embodiment described above, not only measurement values, but also estimated values before an increase or decrease based on the measurement values and the component increase/decrease rate may be output as the measurement result. FIG. 16 illustrates an example of a measurement result screen including the measurement result for the component increase/decrease item according to a second modified example, and FIG. 16 corresponds to FIG. 11. As illustrated in FIG. 16, four measurement items, measurement values respectively corresponding to the four measurement items, two estimated values before an increase or decrease, and information about the component increase/decrease rate are displayed as a measurement result IF5b on a measurement result screen S4b. These measurement values are actually measured values. These estimated values before an increase or decrease are values calculated by dividing the actually measured value by the component increase/decrease rate, and are estimated values before an increase or decrease of a component over time. In the example illustrated in FIG. 16, each of the measurement item "XXX" and the measurement item "YYY" corresponds to the component increase/decrease item for which the flag is set. Accordingly, in a remarks column of the measurement result screen S4b, "increase/decrease guideline: 81%" is displayed as a guideline value for the component increase/decrease rate corresponding to the measurement item "XXX", and the "increase/decrease guideline: 102%" is displayed as a guideline value for the component increase/decrease rate corresponding to the measurement item "YYY" as the information about the component increase/decrease rate, and the estimated value "100" before an increase or decrease of the measurement item "XXX" and the estimated value "97.6" before an increase or decrease of the measurement item "YYY" are also displayed. The user can confirm the measurement values and the estimated values before an increase or decrease, which leads to a reduction in the probability that an increase or decrease in a measurement target component over time affects a diagnosis.

### Modified Examples of First and Second Embodiments

While the automatic analysis apparatuses 1 according to the first and second embodiments are described to be applied to an automatic analysis apparatus that executes a biochemical examination, the first and second embodiments are not limited thereto. More specifically, the automatic analysis apparatuses 1 according to the first and second embodiments can also be applied to an automatic analysis apparatus that executes a blood coagulation analysis examination and an examination using immunoassay.

In the automatic analysis apparatuses 1 according to the first and second embodiments described above, the flag setting function 92 sets the flag for the component increase/decrease item based on an input operation by the user. However, the flag setting function 92 is not limited thereto. For example, the flag setting function 92 may set the flag for the component increase/decrease item based on a previous measurement result for the measurement item. Specifically, the flag setting function 92 may identify a measurement item whose measurement target component increases or decreases over time, and may set the flag for the identified measurement item, thereby setting the flag for the component increase/decrease item.

In the automatic analysis apparatuses 1 according to the first and second embodiments described above, the threshold setting function 93 sets the first threshold for the component increase/decrease item for which the flag is set based on an input operation by the user. However, the threshold setting function 93 is not limited thereto. For example, the threshold setting function 93 may set the first threshold for the component increase/decrease item for which the flag is set based on the previous measurement result for the measurement item. Specifically, the threshold setting function 93 may identify the measurement item a measurement target component thereof increases or decreases over time, and may identify the time until an increase or decrease of the measurement target component affects the measurement result for the identified measurement item to thereby set the first threshold for the component increase/decrease item for which the flag is set.

The term "processor" used in the above description refers to, for example, circuitry such as a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)). The processor implements a function by reading and executing a program stored in storage circuitry. Instead of storing the program in the storage circuitry, the program may be directly incorporated in circuitry of the processor. In this case, the processor implements the function by reading and executing the program incorporated in the circuitry. The processor may be configured with a single piece of circuitry, or may be configured with a plurality of pieces of independent circuitry combined together as one processor to implement the function. Further, a plurality of components may be integrated into one processor to implement functions thereof.

According to at least one of the embodiments described above, it is possible to reduce the probability that an increase or decrease in a measurement target component overtime affects a diagnosis.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An automatic analysis apparatus configured to analyze a mixture of a sample and a reagent corresponding to a measurement item for the sample by using the sample and the reagent, the automatic analysis apparatus comprising processing circuitry configured to:
set a flag for a component increase/decrease item, the flag indicating that the item is a component increase/decrease item, the component increase/decrease item being a measurement item in which a measurement target component in the sample increases or decreases over time; and
set a first threshold for the component increase/decrease item for which the flag is set, the first threshold relating to a time until an increase or decrease of the measurement target component affects a measurement result.

2. The automatic analysis apparatus according to claim 1, wherein the processing circuitry is further configured to:
acquire time information including a time being at least one of a collection time, a measurement request time, and an initial measurement time; and
calculate an elapsed time from the time included in the time information to a current time.

3. The automatic analysis apparatus according to claim 2, wherein the processing circuitry is further configured to set a formula for calculating a component increase/decrease rate relating to a ratio of increase or decrease of the measurement target component based on the elapsed time calculated by the processing circuitry, for the component increase/decrease item for which the flag is set.

4. The automatic analysis apparatus according to claim 3, wherein the processing circuitry is further configured to set an environmental condition for the formula, the environmental condition being a condition relating to an environment in which the sample is stored.

5. The automatic analysis apparatus according to claim 2, wherein the processing circuitry is further configured to determine whether the elapsed time calculated by the processing circuitry has exceeded the first threshold.

6. The automatic analysis apparatus according to claim 2, wherein the processing circuitry is further configured to set a mode for executing measurement of the component increase/decrease item in a case where the elapsed time has exceeded the first threshold.

7. The automatic analysis apparatus according to claim 6, wherein the processing circuitry sets, as the mode for executing the measurement of the component increase/decrease item, for the component increase/decrease item for which the flag is set, one of a measurement execution mode for executing the measurement of the component increase/decrease item even in a case where the elapsed time has exceeded the first threshold, a measurement determination mode for allowing a user to determine whether to measure the component increase/decrease item in the case where the elapsed time has exceeded the first threshold, and a measurement cancelation mode for canceling the measurement of the component increase/decrease item in the case where the elapsed time has exceeded the first threshold.

8. The automatic analysis apparatus according to claim 4, wherein the processing circuitry is further configured to set a second threshold for the formula, the second threshold relating to the component increase/decrease rate.

9. The automatic analysis apparatus according to claim 7, wherein the processing circuitry is further configured to control a display unit to display a measurement availability selection screen for allowing the user to select whether the measurement is possible in a case where the measurement determination mode is set for the component increase/decrease item and the elapsed time has exceeded the first threshold.

10. The automatic analysis apparatus according to claim 9, wherein the processing circuitry is further configured to:
input environmental information to the measurement availability selection screen, the environmental information being information about an environment in which the sample is stored; and
output the input environmental information as a measurement result.

11. The automatic analysis apparatus according to claim 3, wherein the processing circuitry is further configured to output at least one of the elapsed time and the component increase/decrease rate calculated by the processing circuitry in association with a measurement result for the measurement target component in a case where the elapsed time has exceeded the first threshold.

12. The automatic analysis apparatus according to claim 7, wherein the processing circuitry is further configured to notify the user of cancellation of measurement of the component increase/decrease item in a case where the measurement cancelation mode is set for the component increase/decrease item and the elapsed time has exceeded the first threshold.

13. The automatic analysis apparatus according to claim 5, wherein the processing circuitry is further configured to control a display unit to display a setting screen including at least the flag and the first threshold.

14. The automatic analysis apparatus according to claim 13, wherein the setting screen further includes the time information.

15. The automatic analysis apparatus according to claim 14, wherein the setting screen further includes a determination result from the processing circuitry, the determination result indicating whether the elapsed time has exceeded the first threshold.
